(19) United States
(12) Patent Application Publication
(10) Pub. No.: US 2003/0045502 A1
(43) Pub. Date: Mar. 6, 2003

(54) Oral intake solution

(76) Inventors: Masumi Kataoka, Osaka (JP); Yumiko Gondo, Akashi-shi (JP); Shinkatsu Morisawa, Kyoto-shi (JP)

Correspondence Address: BIRCH STEWART KOLASCH & BIRCH, PO BOX 747, FALLS CHURCH, VA, 22040-0747, US

(21) Appl. No.: 10/132,180

(22) Filed: Apr. 26, 2002

(30) Foreign Application Priority Data
May 17, 2001 (JP) 2001-147949

(51) Int. Cl. A61K 33/00; A61K 31/714; A61K 31/51

(52) U.S. Cl. 514/52; 514/276; 424/600

(57) ABSTRACT

An oral intake solution reduced in bitterness and thus easy to administer orally is produced by dissolving a water-soluble component giving a bitter taste, e.g., vitamin B group, into reduced water obtained, e.g., by electrolysis. The pH value of the solution is preferably adjusted to from 2 to 7.

### BACKGROUND OF THE INVENTION

1. Field of the Invention

The present invention relates to a method for reducing a bitter taste and an oral intake solution with reduced bitterness.

2. Description of the Background Art

Vitamin B group is useful for mammals. Its oral administration, however, is difficult due to the characteristic bitter taste. Conventional vitamin B-containing oral intake solutions have sucrose, honey or the like blended thereto, to reduce the bitterness to make it easier to take orally. Still, they cannot eliminate the bitter taste completely.

Various techniques have been developed to reduce such bitterness. Japanese Patent Laying-Open No. 5-4921 discloses a vitamin B-containing oral solution agent blended with amino acids and apple flavor. Japanese Patent Laying-Open No. 5-255126 discloses a composition prepared by combining essential oil or component thereof. Japanese Patent Laying-Open No. 9-328429 discloses blending of licorice extract and nonionic surfactant to the vitamin B₁ derivative. Japanese Patent Laying-Open No. 11-209266 discloses an oral intake solution containing thiamine or its salt, added with a ginger group flavor.

However, there are unfulfilled demands for novel techniques that can reduce bitterness of a water-soluble component like vitamin B group and others.

### SUMMARY OF THE INVENTION

An object of the present invention is to provide a novel method for reducing a bitter taste and an oral intake solution with reduced bitterness.

To achieve such an object, the inventors have found through various studies that electrolytic reduced water (i.e., reduced water obtained by electrolysis) is effective to reduce bitterness of vitamin B group, and have reached the present invention.

More specifically, a method for reducing bitterness according to an aspect of the present invention is characterized in that a water-soluble component giving a bitter taste is dissolved in reduced water. Here, the water-soluble component giving the bitter taste is preferably vitamin B group. The reduced water is preferably electrolytic reduced water.

An oral intake solution according to another aspect of the present invention contains a water-soluble component giving a bitter taste and reduced water. Here, the water-soluble component giving the bitter taste is preferably vitamin B group, and the reduced water is preferably electrolytic reduced water. Further, the oral intake solution preferably has a pH value in a range from 2 to 7.

A method for producing an oral intake solution according to a further aspect of the present invention is characterized in that a water-soluble component giving a bitter taste is dissolved in reduced water.

The foregoing and other objects, features, aspects and advantages of the present invention will become more apparent from the following detailed description of the present invention.

### DESCRIPTION OF THE PREFERRED EMBODIMENTS

The water-soluble component giving a bitter taste to be used in the present invention may be any of those that dissolve into water and leave bitterness in oral cavities after administered, whose solubility and others are not limited specifically. A preferable example of such a component is vitamin B group. Herein, the vitamin B group refers to compounds belonging to the vitamin B group, and their derivatives and pharmaceutically acceptable salts. Specifically, the vitamin B group includes: thiamine; thiamine derivatives (e.g., prosultiamine, fursultiamine, octotiamine, allithiamine, thiamine disulfide, O-benzoyl thiamine disulfide, thiamine mono-phosphate disulfide, O,S-dibenzoyl thiamine, S-benzoyl thiamine, benfotiamine, dicethiamine, diclocarbothiamine) and their pharmaceutically acceptable salts (e.g., acid-added salts including: hydrochloride such as thiamine hydrochloride, nitrate such as thiamine nitrate, phosphate, and sulfate); riboflavin; riboflavin derivatives (e.g., riboflavin butyrate, sodium riboflavin phosphate); pyridoxine; pharmaceutically acceptable salts of pyridoxine (e.g., pyridoxine hydrochloride); pyridoxine derivatives (e.g., pyridoxal phosphate, pyridoxamine phosphate); and, as vitamin B₁₂, cyanocobalamin, hydroxocobalamin, hydroxocobalamin acetate, methylcobalamin. These may be used alone, or two or more of them may be employed together.

The reduced water for use in the present invention refers to water having strong reduction power compared to general tap water and mineral water. It has an oxidation potential of preferably less than 0 mV, more preferably not greater than −100 mV, and most preferably not greater than −500 mV. The reduced water may be produced by subjecting tap water to electrolysis, magnetizing process, electronic process, ultrasonic process, crystal or mineral process (e.g., tourmaline, biotite monzonite spotted stone (bakuhanseki), quartz diorite porphyrite (iouseki)), or other process. Alternatively, the reduced water present in nature may be employed. In the present invention, the reduced water is preferably produced by subjecting electrolyte-containing water to electrolysis; the water thus produced is suitably used for the invention. Herein, the reduced water obtained by electrolysis is called “electrolytic reduced water”.

More specifically, the electrolytic reduced water may be produced by subjecting purified water containing 0.001-0.01% sodium chloride to strong decomposition employing a commercially available, running-water type electrolyzer (TI-8000 model, manufactured by NIHON TRIM CO., LTD.).

The reduced water contains dissolved hydrogen (molecular hydrogen). From the standpoint of effectively masking the bitter taste, the content of the dissolved hydrogen has its lower limit of preferably 400 ppb, more preferably 880 ppb, and its upper limit of preferably 1100 ppb, more preferably 1060 ppb. The concentration of the dissolved hydrogen may be measured using a commercially available measurement device (e.g., DHDI-1 model, manufactured by DKK-TOA Corporation).

According to the method of the present invention, a bitter taste of the water-soluble component giving bitterness can be lessened by dissolving the component into the reduced water. For the dissolution of the component, any known method may be employed. Although the component may be blended in any amount within a range ensuring reduction of the bitterness, in the case of vitamin B group, the component is blended preferably 0.001-0.05 parts by weight, more preferably 0.002-0.04 parts by weight, with respect to 1 part by weight of the electrolytic reduced water.

The oral intake solution of the present invention can be produced according to the inventive bitterness-reducing method. In the case where the water-soluble component giving the bitter taste is vitamin B group, the inventive solution has a pH value preferably in a range from 2 to 7, more preferably in a range from 2.5 to 6.5, from the standpoint of the flavor. The pH value out of the range may affect stability of the vitamin B group. The pH value may be adjusted by adding acid or alkali according to any known method. Although the acid and alkali are unspecified as long as they are pharmaceutically acceptable, the acid may be the same one as listed below being added for the purpose of reduction of bitterness. In this case, the relevant acid works both for adjustment of the pH value and for reduction of the bitter taste.

A sweetening agent may be added to the solution of the present invention to further reduce the bitterness. The ingredient(s) and blended amount(s) thereof may be determined according to any know method. Examples of the sweetening agent include: sugar, fruit sugar, glucose or grape sugar, maltose, liquid sugar, fruit-grape-liquid sugar, grape-fruit-liquid sugar, invert-type liquid sugar, trehalose, palatinose, maltitol, sorbitol, palatinit, erythritol, xylitol, sormatine, sucrose, and stevia extracted refined material. They may be employed alone, or two or more of them may be employed together.

Organic acid or inorganic acid may further be blended to the solution of the present invention, which will further reduce the bitterness. The organic acid may include citric acid, DL-malic acid, tartaric acid, lactic acid, glutamic acid, and aspartic acid. The inorganic acid may include hydrochloric acid and phosphoric acid. Among them, citric acid, DL-malic acid and phosphoric acid are most preferable. They may be employed alone, or two or more of them may be employed together.

Besides the components described above, those generally usable for the oral intake solutions may be blended into the solution of the present invention as desired. The ingredient(s) and blended amount(s) thereof may be determined according to any known method. Such components include: aminoethylsulfonic acid, aspartic acid, arginine, lysine, ascorbic acid, nicotinic acid amide, vitamin A or its derivative, vitamin E or its derivative, carnitine chloride, chondroitin sulfate, caffeine, crude drugs (e.g., ginseng, Eucommia ulmoides, Lurong or young deer horn, cinnamon or cassia bark, guarana or Paullinia cupana, peony or Paeonia albiflora, Jujube or zizyphi fructus, bread or ginger, glycyrrhiza or Glycyrrhiza glabra, hippocampus, cistanchis herba, morindae radix, astragali radix, angericae radix or Japanese angelica root, hoelen or Pachyma hoelen, atractylodis rhizoma, royal jelly) and other components useful for organisms; solutions such as propylene glycol; and preservatives. They may be employed alone, or two or more of them may be employed together.

The oral intake solution of the present invention may be administered in the same manner as a common solution administered orally.

### EXAMPLES

Hereinafter, the present invention will be described in more detail with reference to examples and comparative examples, although they should not be taken by way of limitation.

### Example 1

| | | |
|---|---|---|
| fursultiamine hydrochloride | 2.5 | mg |
| riboflavin | 2 | mg |
| pyridoxine hydrochloride | 10 | mg |
| nicotinic acid amide | 25 | mg |
| sodium L-aspartate | 125 | mg |
| aminoethyl sulfonate | 1000 | mg |
| anhydrous caffeine | 50 | mg |
| citric acid | 350 | mg |
| tartaric acid | 100 | mg |
| DL-malic acid | 100 | mg |
| monosodium L-glutamate | 5 | mg |
| purified sucrose | 9 | g |
| reduced maltose starch syrup | 1.5 | g |
| sodium benzoate | 60 | mg |
| ethyl parahydroxybenzoate | 6 | mg |

| | |
|---|---|
| flavoring agent | infinitesimal dose |
| electrolytic reduced water | added to 100 mL |

The ingredients above were mixed and dissolved into the water, which was filtered by a membrane filter of 0.45 μm to obtain a test solution. The pH value of the solution was from 2.9 to 3.1.

### Example 2

| | | |
|---|---|---|
| fursultiamine hydrochloride | 5 | mg |
| riboflavin | 2 | mg |
| pyridoxine hydrochloride | 10 | mg |
| nicotinic acid amide | 25 | mg |
| sodium L-aspartate | 125 | mg |
| aminoethyl sulfonate | 1500 | mg |
| anhydrous caffeine | 50 | mg |
| citric acid | 350 | mg |
| tartaric acid | 100 | mg |
| DL-malic acid | 100 | mg |
| monosodium L-glutamate | 5 | mg |
| sodium chloride | 40 | mg |
| purified sucrose | 8 | g |
| fruit sugar | 2 | g |
| reduced maltose starch syrup | 1 | g |
| sodium benzoate | 60 | mg |
| ethyl parahydroxybenzoate | 6 | mg |

| | |
|---|---|
| flavoring agent | infinitesimal dose |
| electrolytic reduced water | added to 100 mL |

The ingredients above were mixed and dissolved into the water, which was filtered by a membrane filter of 0.45 μm to obtain a test solution. The pH value of the solution was from 2.9 to 3.1.

### Example 3

| | | |
|---|---|---|
| fursultiamine hydrochloride | 5 | mg |
| riboflavin | 2 | mg |
| pyridoxine hydrochloride | 10 | mg |
| nicotinic acid amide | 25 | mg |
| sodium L-aspartate | 125 | mg |
| aminoethyl sulfonate | 1500 | mg |
| anhydrous caffeine | 50 | mg |
| citric acid | 350 | mg |
| tartaric acid | 100 | mg |
| DL-malic acid | 100 | mg |
| monosodium L-glutamate | 5 | mg |
| sodium chloride | 40 | mg |
| purified sucrose | 8 | g |
| fruit sugar | 2 | g |
| powder of reduced maltose starch syrup | 1 | g |
| sodium benzoate | 60 | mg |
| ethyl parahydroxybenzoate | 6 | mg |

| | |
|---|---|
| flavoring agent | infinitesimal dose |
| electrolytic reduced water | added to 100 mL |

The ingredients above were mixed and dissolved into the water, which was filtered by a membrane filter of 0.45 μm to obtain a test solution. The pH value of the solution was from 2.9 to 3.1.

### Example 4

| | | |
|---|---|---|
| fursultiamine hydrochloride | 5 | mg |
| riboflavin | 2 | mg |
| pyridoxine hydrochloride | 10 | mg |
| nicotinic acid amide | 25 | mg |
| sodium L-aspartate | 125 | mg |
| anhydrous caffeine | 50 | mg |
| citric acid | 150 | mg |
| tartaric acid | 100 | mg |
| DL-malic acid | 50 | mg |
| purified sucrose | 9 | g |
| sodium benzoate | 30 | mg |
| ethyl parahydroxybenzoate | 2.5 | mg |

| | |
|---|---|
| flavoring agent | infinitesimal dose |
| electrolytic reduced water | added to 50 mL |

The ingredients above were mixed and dissolved into the water, which was filtered by a membrane filter of 0.45 μm to obtain a test solution. The pH value of the solution was from 3.0 to 3.2.

### Example 5

| | | |
|---|---|---|
| fursultiamine hydrochloride | 5 | mg |
| sodium riboflavin phosphate | 2 | mg |
| pyridoxine hydrochloride | 10 | mg |
| nicotinic acid amide | 25 | mg |
| sodium L-aspartate | 125 | mg |
| anhydrous caffeine | 50 | mg |
| citric acid | 150 | mg |
| tartaric acid | 100 | mg |
| DL-malic acid | 50 | mg |
| purified sucrose | 9 | g |
| sodium benzoate | 30 | mg |
| butyl parahydroxybenzoate | 2.5 | mg |

| | |
|---|---|
| flavoring agent | infinitesimal dose |
| electrolytic reduced water | added to 50 mL |

The ingredients above were mixed and dissolved into the water, which was filtered by a membrane filter of 0.45 μm to obtain a test solution. The pH value of the solution was from 3.0 to 3.2.

### Example 6

| | | |
|---|---|---|
| fursultiamine hydrochloride | 10 | mg |
| sodium riboflavin phosphate | 5 | mg |
| pyridoxine hydrochloride | 10 | mg |
| nicotinic acid amide | 30 | mg |
| sodium L-aspartate | 125 | mg |
| aminoethyl sulfonate | 1000 | mg |
| carnitine chloride | 100 | mg |
| sodium chondroitin sulfate | 120 | mg |
| anhydrous caffeine | 50 | mg |
| citric acid | 330 | mg |
| sodium citrate | 30 | mg |
| tartaric acid | 50 | mg |
| lactic acid | 0.1 | mL |
| purified sucrose | 7 | g |
| honey | 3.5 | g |
| sodium benzoate | 35 | mg |
| ethyl parahydroxybenzoate | 2.5 | mg |

| | |
|---|---|
| flavoring agent | infinitesimal dose |
| electrolytic reduced water | added to 50 mL |

The ingredients above were mixed and dissolved into the water, which was filtered by a membrane filter of 0.45 μm to obtain a test solution. The pH value of the solution was from 2.9 to 3.1.

### Comparative Example 1

| | | |
|---|---|---|
| fursultiamine hydrochloride | 2.5 | mg |
| riboflavin | 2 | mg |
| pyridoxine hydrochloride | 10 | mg |
| nicotinic acid amide | 25 | mg |
| sodium L-aspartate | 125 | mg |
| aminoethyl sulfonate | 1000 | mg |
| anhydrous caffeine | 50 | mg |
| citric acid | 350 | mg |
| tartaric acid | 100 | mg |
| DL-malic acid | 100 | mg |
| monosodium L-glutamate | 5 | mg |
| purified sucrose | 9 | g |
| reduced maltose starch syrup | 1.5 | g |
| sodium benzoate | 60 | mg |
| ethyl parahydroxybenzoate | 6 | mg |

| | |
|---|---|
| flavoring agent | infinitesimal dose |
| purified water | added to 100 mL |

The ingredients above were mixed and dissolved into the water, which was filtered by a membrane filter of 0.45 μm to obtain a test solution. The pH value of the solution was from 2.9 to 3.1.

### Comparative Example 2

| | | |
|---|---|---|
| fursultiamine hydrochloride | 5 | mg |
| riboflavin | 2 | mg |
| pyridoxine hydrochloride | 10 | mg |
| nicotinic acid amide | 25 | mg |
| sodium L-aspartate | 125 | mg |
| aminoethyl sulfonate | 1500 | mg |
| anhydrous caffeine | 50 | mg |
| citric acid | 350 | mg |
| tartaric acid | 100 | mg |
| DL-malic acid | 100 | mg |
| monosodium L-glutamate | 5 | mg |
| sodium chloride | 40 | mg |
| purified sucrose | 8 | g |
| fruit sugar | 2 | g |
| reduced maltose starch syrup | 1 | g |
| sodium benzoate | 60 | mg |
| ethyl parahydroxybenzoate | 6 | mg |

| | |
|---|---|
| flavoring agent | infinitesimal dose |
| purified water | added to 100 mL |

The ingredients above were mixed and dissolved into the water, which was filtered by a membrane filter of 0.45 μm to obtain a test solution. The pH value of the solution was from 2.9 to 3.1.

### Comparative Example 3

| | | |
|---|---|---|
| fursultiamine hydrochloride | 5 | mg |
| riboflavin | 2 | mg |
| pyridoxine hydrochloride | 10 | mg |
| nicotinic acid amide | 25 | mg |
| sodium L-aspartate | 125 | mg |
| aminoethyl sulfonate | 1500 | mg |
| anhydrous caffeine | 50 | mg |
| citric acid | 350 | mg |
| tartaric acid | 100 | mg |
| DL-malic acid | 100 | mg |
| monosodium L-glutamate | 5 | mg |
| sodium chloride | 40 | mg |
| purified sucrose | 8 | g |
| fruit sugar | 2 | g |
| powder of reduced maltose starch syrup | 1 | g |
| sodium benzoate | 60 | mg |
| ethyl parahydroxybenzoate | 6 | mg |

| | |
|---|---|
| flavoring agent | infinitesimal dose |
| purified water | added to 100 mL |

The ingredients above were mixed and dissolved into the water, which was filtered by a membrane filter of 0.45 μm to obtain a test solution. The pH value of the solution was from 2.9 to 3.1.

### Comparative Example 4

| | | |
|---|---|---|
| fursultiamine hydrochloride | 5 | mg |
| riboflavin | 2 | mg |
| pyridoxine hydrochloride | 10 | mg |
| nicotinic acid amide | 25 | mg |
| sodium L-aspartate | 125 | mg |
| anhydrous caffeine | 50 | mg |
| citric acid | 150 | mg |
| tartaric acid | 100 | mg |
| DL-malic acid | 50 | mg |
| purified sucrose | 9 | g |
| sodium benzoate | 30 | mg |
| ethyl parahydroxybenzoate | 2.5 | mg |

| | |
|---|---|
| flavoring agent | infinitesimal dose |
| purified water | added to 50 mL |

The ingredients above were mixed and dissolved into the water, which was filtered by a membrane filter of 0.45 μm to obtain a test solution. The pH value of the solution was from 3.0 to 3.2.

### Comparative Example 5

| | | |
|---|---|---|
| fursultiamine hydrochloride | 5 | mg |
| sodium riboflavin phosphate | 2 | mg |
| pyridoxine hydrochloride | 10 | mg |
| nicotinic acid amide | 25 | mg |
| sodium L-aspartate | 125 | mg |
| anhydrous caffeine | 50 | mg |
| citric acid | 150 | mg |
| tartaric acid | 100 | mg |
| DL-malic acid | 50 | mg |
| purified sucrose | 9 | g |
| sodium benzoate | 30 | mg |
| butyl parahydroxybenzoate | 2.5 | mg |

| | |
|---|---|
| flavoring agent | infinitesimal dose |
| purified water | added to 50 mL |

The ingredients above were mixed and dissolved into the water, which was filtered by a membrane filter of 0.45 μm to obtain a test solution. The pH value of the solution was from 3.0 to 3.2.

### Comparative Example 6

| | | |
|---|---|---|
| fursultiamine hydrochloride | 10 | mg |
| sodium riboflavin phosphate | 5 | mg |
| pyridoxine hydrochloride | 10 | mg |
| nicotinic acid amide | 30 | mg |
| sodium L-aspartate | 125 | mg |
| aminoethyl sulfonate | 1000 | mg |
| carnitine chloride | 100 | mg |
| sodium chondroitin sulfate | 120 | mg |
| anhydrous caffeine | 50 | mg |
| citric acid | 330 | mg |
| sodium citrate | 30 | mg |
| tartaric acid | 50 | mg |
| lactic acid | 0.1 | mL |
| purified sucrose | 7 | g |
| honey | 3.5 | g |
| sodium benzoate | 35 | mg |
| ethyl parahydroxybenzoate | 2.5 | mg |

| | |
|---|---|
| flavoring agent | infinitesimal dose |
| purified water | added to 50 mL |

The ingredients above were mixed and dissolved into the water, which was filtered by a membrane filter of 0.45 μm to obtain a test solution. The pH value of the solution was from 2.9 to 3.1.

### Test Example 1

Sensory Evaluation Regarding Bitterness

Ten healthy adults were selected to conduct a sensory test on bitterness. Each specimen was tested and evaluated in four levels of “bitter”, “slightly bitter”, “hardly bitter” and “not bitter”. The specimens were tested at random by each test subject. Table 1 shows the results.

TABLE 1
Result of sensory test on bitterness

| | | | | (unit: person) |
|---|---|---|---|---|
| | | slightly | hardly | not |
| Specimen | bitter | bitter | bitter | bitter |
| Example 1 | 0 | 0 | 0 | 10 |
| Example 2 | 0 | 0 | 0 | 10 |
| Example 3 | 0 | 0 | 0 | 10 |
| Example 4 | 0 | 0 | 1 | 9 |
| Example 5 | 0 | 0 | 2 | 8 |
| Example 6 | 0 | 1 | 7 | 2 |
| Comparative | 0 | 1 | 9 | 0 |
| Example 1 | | | | |
| Comparative | 0 | 4 | 6 | 0 |
| Example 2 | | | | |
| Comparative | 0 | 2 | 8 | 0 |
| Example 3 | | | | |
| Comparative | 0 | 4 | 6 | 0 |
| Example 4 | | | | |
| Comparative | 0 | 3 | 7 | 0 |
| Example 5 | | | | |
| Comparative | 1 | 4 | 5 | 0 |
| Example 6 | | | | |

As seen from Table 1, while almost all the Comparative examples were evaluated as “slightly bitter” or “hardly bitter”, most of the Examples of the present invention were evaluated as “hardly bitter” or “not bitter”.

As such, according to the present invention, an oral intake solution reduced in bitterness of vitamin B group and hence suitable for oral administration is provided.

Although the present invention has been described and illustrated in detail, it is clearly understood that the same is by way of illustration and example only and is not to be taken by way of limitation, the spirit and scope of the present invention being limited only by the terms of the appended claims.

What is claimed is:

1. A method for reducing bitterness characterized in that a water-soluble component presenting a bitter taste is dissolved into reduced water.

2. The method according to claim 1, wherein the water-soluble component presenting the bitter taste is vitamin B group.

3. The method according to claim 1, wherein the reduced water is electrolytic reduced water.

4. An oral intake solution including a water-soluble component presenting a bitter taste and reduced water.

5. The oral intake solution according to claim 4, wherein the water-soluble component presenting the bitter taste is vitamin B group.

6. The oral intake solution according to claim 4, wherein the reduced water is electrolytic reduced water.

7. The oral intake solution according to claim 5, having a pH value in a range from 2 to 7.

8. A method for producing the oral intake solution according to claim 4, characterized in that the water-soluble component presenting the bitter taste is dissolved into the reduced water.
